(19) United States
(12) Patent Application Publication
(10) Pub. No.: US 2025/0177069 A1
(43) Pub. Date: Jun. 5, 2025

(54) SURGICAL ROBOT ARM CONTROL SYSTEM AND SURGICAL ROBOT ARM CONTROL METHOD

(71) Applicant: Metal Industries Research & Development Centre, Kaohsiung (TW)

(72) Inventors: Bo-Wei Pan, Kaohsiung City (TW); Sheng-Hung Yang, Kaohsiung City (TW); Wei Han Hsieh, Kaohsiung City (TW)

(73) Assignee: Metal Industries Research & Development Centre, Kaohsiung (TW)

(21) Appl. No.: 18/528,786

(22) Filed: Dec. 5, 2023

(51) Int. Cl. A61B 34/32; A61B 34/10; A61B 34/20; G05B 19/4155

(52) U.S. Cl. CPC A61B 34/32; G05B 19/4155; A61B 2034/107; A61B 2034/2065; A61B 2034/2072; G05B 2219/45123

(57) ABSTRACT

A surgical robot arm control system and a surgical robot arm control method are provided. The surgical robot arm control system includes a surgical robot arm, a spatial positioning information acquisition unit, a depth image acquisition unit, and a processor. The spatial positioning information acquisition unit is configured to acquire spatial coordinate data. The depth image acquisition unit is configured to acquire a panoramic depth image. The processor performs image recognition on the panoramic depth image to recognize the surgical robot arm and locates a position of the surgical robot arm based on the spatial coordinate data. The processor defines an environmental space according to the position of the surgical robot arm and plans a movement path of the surgical robot arm in the environmental space. The processor controls the surgical robot arm according to the movement path of the surgical robot arm.

### BACKGROUND

### Technical Field

The disclosure relates to a control system; more particularly, the disclosure relates to a surgical robot arm control system and a surgical robot arm control method.

### Description of Related Art

At present, surgical robot arms are widely utilized in diverse medical procedures and aid medical professionals in conducting associated surgical operations. Specifically, these robot arms may be configured to mitigate the risk of unwarranted injuries to a surgical subject resulting from hand tremors of medical personnel during surgery, which may effectively reduce blood loss, minimize wounds, alleviate pain, shorten hospital stays, decrease the likelihood of postoperative infections, and expedite the recovery process for the surgical subject after surgery. However, in the existing applications of surgical robot arm control, medical personnel typically remain responsible for overseeing overall movement and control, thus leading to a potential for operational errors and a reduced operational efficiency.

### SUMMARY

The disclosure provides a surgical robot arm control system and a surgical robot arm control method, which may effectively provide assistance to surgeries.

An embodiment of the disclosure provides a surgical robot arm control system that includes a surgical robot arm, a spatial positioning information acquisition unit, a depth image acquisition unit, and a processor. The spatial positioning information acquisition unit is configured to acquire spatial coordinate data. The depth image acquisition unit is configured to acquire a panoramic depth image. The processor is coupled to the surgical robot arm, the spatial positioning information acquisition unit, and the depth image acquisition unit. The processor performs image recognition on the panoramic depth image to recognize the surgical robot arm and locates a position of the surgical robot arm according to the spatial coordinate data. The processor defines an environmental space according to the position of the surgical robot arm and plans a movement path of the surgical robot arm in the environmental space. The processor controls the surgical robot arm according to the movement path of the surgical robot arm.

An embodiment of the disclosure provides a surgical robot arm control method that includes following steps. Spatial coordinate data are acquired by a spatial positioning information acquisition unit. A panoramic depth image is acquired by a depth image acquisition unit. Image recognition is performed on the panoramic depth image by a processor to recognize a surgical robot arm. A position of the surgical robot arm is located by the processor according to the spatial coordinate data. By the processor, an environmental space is defined according to the position of the surgical robot arm, and a movement path of the surgical robot arm in the environmental space is planned. The surgical robot arm is controlled by the processor according to the movement path of the surgical robot arm.

Based on the above, the surgical robot arm control system and the surgical robot arm control method provided in one or more embodiments of the disclosure may be applied to recognize the environment through computer vision images and perform positioning to automatically control the surgical robot arm to move to the position of the target region.

Several exemplary embodiments accompanied with figures are described in detail below to further describe the disclosure in details.

### BRIEF DESCRIPTION OF THE DRAWINGS

The accompanying drawings are included to provide a further understanding of the disclosure, and are incorporated in and constitute a part of this specification. The drawings illustrate exemplary embodiments of the disclosure and, together with the description, serve to explain the principles of the disclosure.

FIG. 1 is a schematic circuit diagram of a surgical robot arm control system according to an embodiment of the disclosure.

FIG. 2 is a flowchart of a surgical robot arm control method according to an embodiment of the disclosure.

FIG. 3 is a schematic diagram of a plurality of modules according to an embodiment of the disclosure.

FIG. 4 is a schematic diagram of a scenario of operating a surgical robot arm according to an embodiment of the disclosure.

FIG. 5 is a schematic diagram of a scenario of operating a surgical robot arm according to an embodiment of the disclosure.

FIG. 6 is a flowchart of a surgical robot arm control method according to an embodiment of the disclosure.

### DESCRIPTION OF THE EMBODIMENTS

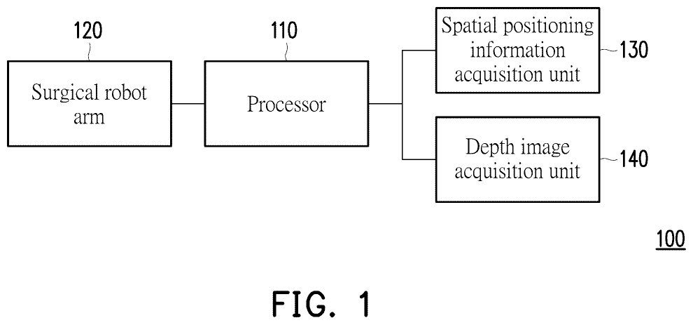

FIG. 1 is a schematic circuit diagram of a surgical robot arm control system according to an embodiment of the disclosure. With reference to FIG. 1, a surgical robot arm control system 100 includes a processor 110, a surgical robot arm 120, a spatial positioning information acquisition unit 130, and a depth image acquisition unit 140. The processor 110 is coupled to the surgical robot arm 120, the spatial positioning information acquisition unit 130, and the depth image acquisition unit 140. In the present embodiment, the surgical robot arm control system 100 may be disposed in an operating room or other surgical environments and may provide assistance during the surgical process conducted by medical personnel.

In the present embodiment, the processor 110 may be disposed in a personal computer (PC), a notebook computer, a tablet, an industrial computer, an embedded computer, a cloud server, and so on, for instance.

In electronic devices with computational capabilities are applicable, which should not be construed as a limitation in the disclosure. In this embodiment, the surgical robot arm 120 may include at least three joint axes to achieve a robot arm with six degrees of freedom in space, for instance. The processor 110 may control the surgical robot arm 120 and implement both forward and inverse kinematics of the robot arm.

In this embodiment, the spatial positioning information acquisition unit 130 may be a camcorder or camera device and may serve to obtain spatial coordinate data. In this embodiment, the depth image acquisition unit 140 may be a depth camera and is configured to generate a panoramic depth image, where the panoramic depth image may include, for instance, RGB digital image information and/or depth image information.

In this embodiment, the surgical robot arm control system 100 may further include a display (not shown in the drawings). The processor 110 is coupled to the display. In this embodiment, the surgical robot arm control system 100 may further include a storage device (not shown in the drawings). The processor 110 is coupled to the storage device. The storage device may include a memory, where the memory may be, for instance, a non-volatile memory, a volatile memory, a hard disc drive, a semiconductor memory, or the like. The non-volatile memory includes, for instance, a read-only memory (ROM), an erasable programmable read-only memory (EPROM), and any other non-volatile memory, and the volatile memory includes a random-access memory (RAM). The memory serves to store various modules, images, information, parameters, and data provided in the disclosure.

In this embodiment, the processor 110 may connect the surgical robot arm 120 through an internet protocol (IP), a universal serial bus (USB), a type-C USB, and so on, and the processor 110 may execute a robot arm automatic control module to control the surgical robot arm 120.

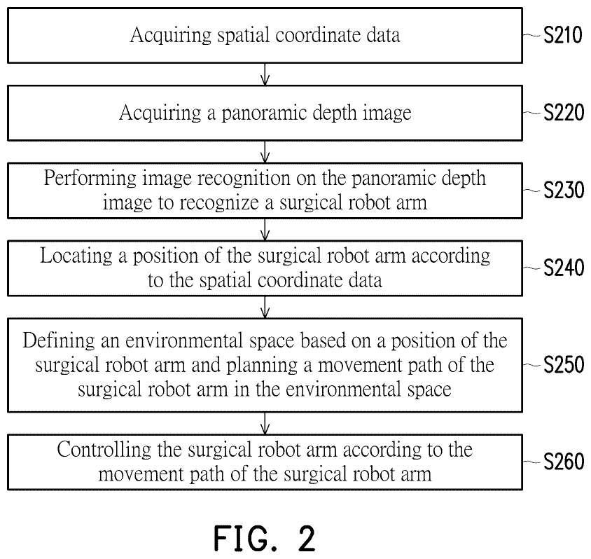

FIG. 2 is a flowchart of a surgical robot arm control method according to an embodiment of the disclosure. With reference to FIG. 1 and FIG. 2, the surgical robot arm control system 100 may execute following steps S210-S260. In step S210, the spatial positioning information acquisition unit 130 may acquire spatial coordinate data. In the present embodiment, the spatial positioning information acquisition unit 130 may detect position information (e.g., coordinates) of each object within the acquisition range. In step S220, the depth image acquisition unit 140 may acquire a panoramic depth image. In the present embodiment, the panoramic depth image may include images of at least one tracking ball, a surgical subject, and the surgical robot arm 120, and the at least one tracking ball may be disposed on (attached to) the surgical subject. It is of significance to highlight that the tracking ball is configured to be attached to a surgical instrument, which is situated on the surgical subject. Consequently, the surgical robot arm control system 100 is capable of recognizing the position of the surgical instrument, and a movement path of the surgical robot arm 120 may be adjusted to navigate around the surgical instrument and thereby prevent potential collisions. The tracking ball may be, for instance, a polyhedron ball and include a positioning pattern, so as to facilitate the spatial positioning information acquisition unit 130 to perform positioning; however, the form of the tracking ball should not be construed as a limitation in the disclosure.

In step S230, the processor 110 may perform image recognition on the panoramic depth image to recognize the surgical robot arm 120. In step S240, the processor 110 may locate a position of the surgical robot arm 120 according to the spatial coordinate data. In the present embodiment, the processor 110 may perform the image recognition on the panoramic depth image to recognize the at least one tracking ball, the surgical subject, and the surgical robot arm 120. The spatial coordinate data include a plurality of coordinates of the at least one tracking ball, the surgical subject, and the surgical robot arm 120.

In step S250, the processor 110 may define the environmental space based on the position of the surgical robot arm 120 and plan the movement path of the surgical robot arm in the environmental space. In step S260, the processor 110 may control the surgical robot arm 120 according to the movement path of the surgical robot arm. The environmental space is a regional range in the real space. In the present embodiment, the environmental space may be centered around an end mechanism of the surgical robot arm 120, and the environmental space is updated together with a movement of the end mechanism of the surgical robot arm 120, which should however not be construed as a limitation in the disclosure. In the present embodiment, the processor 110 may train a real path model corresponding to the environmental space through transfer learning according to a virtual path model, so as to acquire the movement path of the surgical robot arm through the real path model. In the present embodiment, the virtual path model and the real path model are respectively a densely connected convolutional network (densenet) model, which should however not be construed as a limitation in the disclosure. In an embodiment, the virtual path model and the real path model may also be other types of convolutional neural network models.

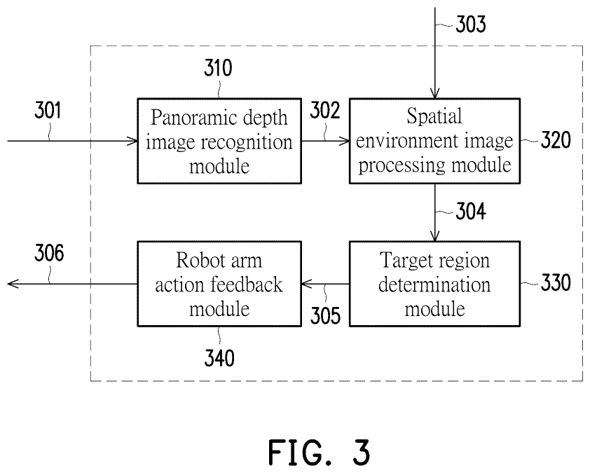

FIG. 3 is a schematic diagram of a plurality of modules according to an embodiment of the disclosure. With reference to FIG. 1 and FIG. 3, a storage device of the surgical robot arm control system 100 may store relevant algorithms and/or programs of a panoramic depth image recognition module 310, a spatial environment image processing module 320, a target region determination module 330, and a robot arm action feedback module 340 as shown in FIG. 3, and the processor 110 may execute the relevant algorithms and/or programs. Specifically, the panoramic depth image recognition module 310 may acquire a panoramic depth image 301, which includes image content of an environmental field, image information, depth information, and direction information. The panoramic depth image recognition module 310 may recognize obstacles (not necessarily present), at least one tracking ball, a surgical subject, and the surgical robot arm 120 in the panoramic depth image 301 and output relevant depth image information 302 to the spatial environment image processing module 320.

The spatial environment image processing module 320 may acquire the relevant depth image information 302 and spatial coordinate data 303. The spatial coordinate data 310 include three-dimensional spatial coordinate values (i.e., providing relevant parameters of the world coordinate system). The spatial coordinate data 310 include a plurality of coordinates of the at least one tracking ball, the surgical subject, and the surgical robot arm 120. The spatial environment image processing module 320 may take the position of an end mechanism (such as a robot claw) of the surgical robot arm 120 as a center point and acquire local image content from the panoramic depth image 301. The spatial environment image processing module 320 may extend from this center point to the surrounding space to form an environmental space matrix that includes this center point. It is worth noting that as this center point moves, the environmental space is updated together with the movement of the end mechanism of the surgical robot arm 120. The processor 110 may automatically generate the movement path of the surgical robot arm based on the spatial position information of the obstacles (if any), the at least one tracking ball, the surgical subject, and the end mechanism of the surgical robot arm 120 in this environmental space. As such, the surgical robot arm 120 does not collide with obstacles (if any), the at least one tracking ball, and the surgical subject on this movement path of the surgical robot arm. The spatial environment image processing module 320 may provide a target coordinate point 304 of the surgical robot arm 120 in the movement path of the surgical robot arm to the target region determination module 330.

Before the surgical robot arm 120 is moved, the processor 110 may execute the target region determination module 330 to re-define the target coordinate point, thus extending the target coordinate point to a line segment and then converting the line segment into a reference target region. The target region determination module 330 may determine whether the reference target region matches the target region to decide whether to control the surgical robot arm according to the movement path of the surgical robot arm. The target region determination module 330 may provide a determination result 305 (i.e., the determined target coordinate point) to the robot arm action feedback module 340. The processor 110 may generate related robot arm control instructions based on the determined target coordinate point, and the robot arm action feedback module 340 may generate a driving signal 306 according to related robot arm control instructions and output the driving signal 306 to the surgical robot arm 120 to drive the surgical robot arm 120.

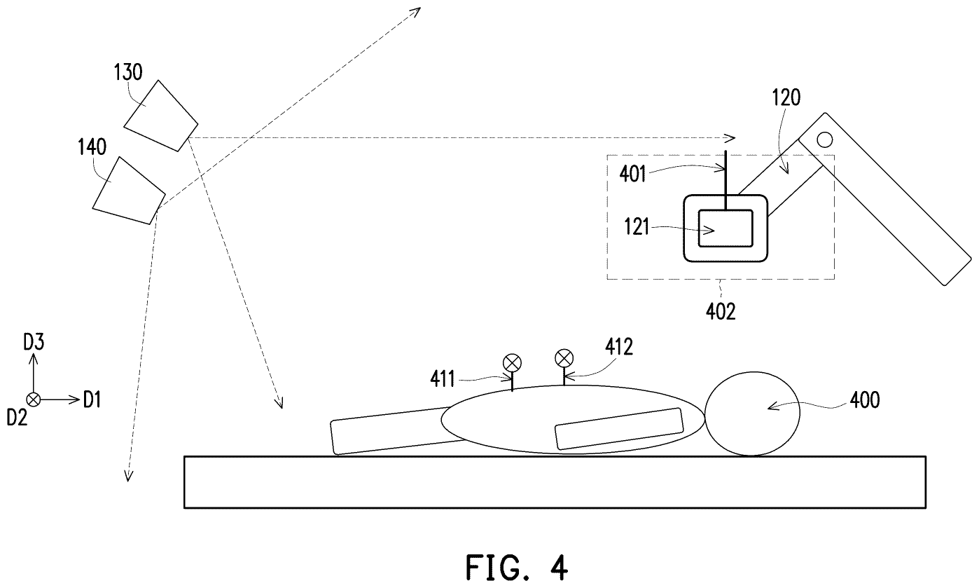

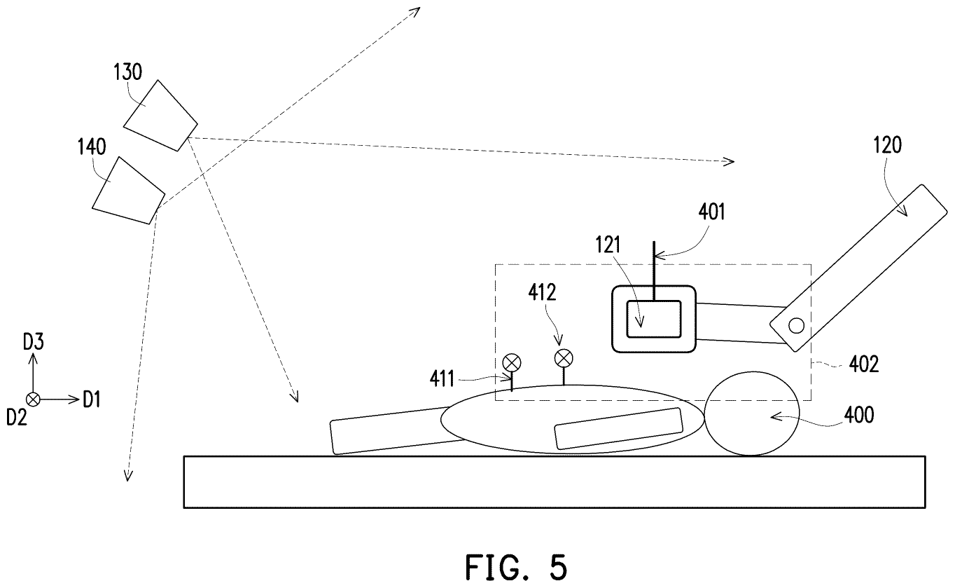

FIG. 4 is a schematic diagram of a scenario of operating a surgical robot arm according to an embodiment of the disclosure. FIG. 5 is a schematic diagram of a scenario of operating a surgical robot arm according to an embodiment of the disclosure. For instance, with reference to FIG. 1, FIG. 4, and FIG. 5, a scenario involving a medical professional engaged in a pre-surgical vertebral drilling operation within the realm of orthopedic medicine is taken as an example. In FIG. 4, a surgical subject 400 (i.e., a patient) is positioned in a prone orientation on an operating table. A surface of the operating table aligns parallel to a plane defined by an extension of direction D1 (a horizontal direction) and a direction D2 (a horizontal direction). A direction D3 signifies a vertical direction. In the present embodiment, the end mechanism 121 of the surgical robot arm 120 may, for instance, secure a surgical instrument 401.

In the present embodiment, the spatial positioning information acquisition unit 130 may acquire the spatial coordinate data of each object in the surgical environment. The depth image acquisition unit 140 may acquire a panoramic depth image of the surgical environment. An acquisition angle of the depth image acquisition unit 140 is greater than an acquisition angle of the spatial positioning information acquisition unit 130. The processor 110 may acquire the spatial coordinates of the surgical subject 400, the tracking balls 411 and 412 of the surgical instrument disposed on the surgical subject 400, the surgical robot arm 120, and the end mechanism 121 of the surgical robot arm 120 in the real world, and the processor 110 may define an environmental space 402 (a cubic region) centered around the end mechanism 121 of the surgical robot arm 120. The processor 110 may train a real path model corresponding to the environmental space 402 based on a virtual path model through transfer learning, so as to acquire the movement path of the surgical robot arm in the environmental space 402 through the real path model. In addition, the end mechanism 121 of the surgical robot arm 120 may also be equipped with a reference tracking ball, and the processor 110 may accurately locate a position of the end mechanism 121 of the surgical robot arm according to the reference tracking ball.

In FIG. 5, the processor 110 may gradually control the end mechanism 121 of the surgical robot arm 120 to approach the surgical subject 400. During the movement of the end mechanism 121 of the surgical robot arm 120, the end mechanism 121 of the surgical robot arm 120 may be adjusted to effectively navigate around the surgical subject 400 and the tracking balls 411 and 412 to prevent potential collisions. Moreover, when the processor 110 determines through the spatial positioning information acquisition unit 130 that the end mechanism 121 of the surgical robot arm 120 reaches the target region, the processor 110 may stop moving and fix the end mechanism 121 of the surgical robot arm 120, so that medical personnel may conveniently use or pick up the surgical instrument 401 to perform surgery on the surgical subject 400.

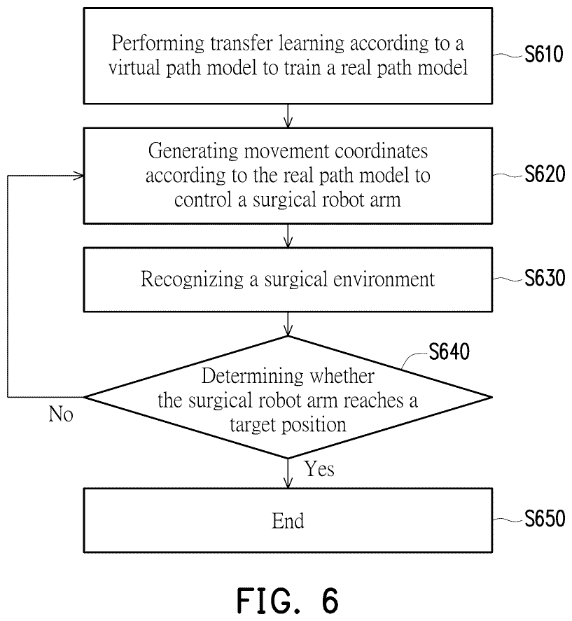

FIG. 6 is a flowchart of a surgical robot arm control method according to an embodiment of the disclosure. With reference to FIG. 1 and FIG. 6, the surgical robot arm control system 100 may execute following steps S610-S650. In step S610, the processor 110 may perform the transfer learning according to the virtual path model to train the real path model. In the present embodiment, the processor 110 may first establish a virtual surgical environment model. The virtual surgical environment model may include, for instance, a virtual surgical subject, a virtual spine model, and a virtual surgical robot arm. The virtual spine model is disposed at a predetermined position in the virtual surgical subject. In the present embodiment, the virtual surgical environment model may be software, e.g., V-Rep or MuJoCo, and may allow the placement of the virtual surgical robot arm, the virtual spine model, virtual identification objects, or the like in the virtual environment. The processor 110 may train a virtual movement path of the virtual surgical robot arm in the virtual surgical environment to establish the virtual path model and may employ a prototype conversion technology for the relocation between the virtual and real surgical environments, utilizing the transfer learning for feature weight transfer. This process aims to align the panoramic depth image and the spatial coordinate data of each object, facilitating the establishment of the real path model. Specifically, the processor 110 has the capability to substitute the feature weights of the virtual path model with those of the real path model and subsequently generate updated feature weights. Besides, the processor 110 may introduce a randomized spectrum of feature weight differences into a reward mechanism of the model for validation purposes. The decision to replace the feature weights is determined based on whether the resulting reward value is maximal. This approach effectively mitigates the blurring of original features across the entire convolution layer of the real path model, enhancing feature segmentation and promoting effective generalization to the actual spatial context of the spinal surgery.

In step S620, the processor 110 may generate movement coordinates according to the real path model to control the surgical robot arm 120. In step S630, after the surgical robot arm 120 is moved, the processor 110 may recognize a surgical environment surrounding the surgical robot arm 120. In step S640, the processor 110 may determine whether the surgical robot arm 120 reaches a target position (i.e., an end component of the surgical robot arm 120 is located at the target coordinate point). If not, the processor 110 may re-define a new environmental space through the real path model or by re-training the real path model, and the processor 110 may plan the movement path of the surgical robot arm in the new environmental space. If yes, the processor 110 may end the movement operation of the surgical robot arm 120 to stop and fix the end mechanism of the surgical robot arm 120, so that medical personnel may conveniently use or pick up the surgical instrument held by the end mechanism to perform surgery on the surgical subject.

To sum up, the surgical robot arm control system and the surgical robot arm control method provided in one or more of the disclosure may be applied to recognize relevant effective environmental space features and obstacle locations through computer vision images and machine learning algorithms. By deducing a plurality of directions to navigate around the obstacles and selecting an optimal path, the surgical robot arm may operate in an inverse kinematics mode, which enables the robot arm to automatically circumvent objects related to the environment during the end displacement process, ultimately reaching the target region.

It will be apparent to those skilled in the art that various modifications and variations can be made to the disclosed embodiments without departing from the scope or spirit of the disclosure. In view of the foregoing, it is intended that the disclosure covers modifications and variations provided that they fall within the scope of the following claims and their equivalents.

What is claimed is:

1. A surgical robot arm control system, comprising:
a surgical robot arm;
a spatial positioning information acquisition unit, configured to acquire spatial coordinate data;
a depth image acquisition unit, configured to acquire a panoramic depth image; and
a processor, coupled to the surgical robot arm, the spatial positioning information acquisition unit, and the depth image acquisition unit,
wherein the processor performs image recognition on the panoramic depth image to recognize the surgical robot arm and locates a position of the surgical robot arm according to the spatial coordinate data, wherein the processor defines an environmental space according to the position of the surgical robot arm and plans a movement path of the surgical robot arm in the environmental space,
wherein the processor controls the surgical robot arm according to the movement path of the surgical robot arm.

2. The surgical robot arm control system according to claim 1, wherein the panoramic depth image comprises at least one tracking ball and an image of the surgical robot arm, and the at least one tracking ball is disposed on a surgical subject,
wherein the processor performs the image recognition on the panoramic depth image to recognize the at least one tracking ball, the surgical subject, and the surgical robot arm, and the spatial coordinate data comprise a plurality of coordinates of the at least one tracking ball, the surgical subject, and the surgical robot arm.

3. The surgical robot arm control system according to claim 1, wherein the environmental space is centered around an end mechanism of the surgical robot arm, and the environmental space is updated together with a movement of the end mechanism of the surgical robot arm.

4. The surgical robot arm control system according to claim 1, wherein the processor trains a real path model corresponding to the environmental space based on a virtual path model through transfer learning to acquire the movement path of the surgical robot arm through the real path model.

5. The surgical robot arm control system according to claim 4, wherein the virtual path model and the real path model are respectively a densely connected convolutional network model.

6. The surgical robot arm control system according to claim 1, wherein after the surgical robot arm is moved, the processor recognizes a surgical environment around the surgical robot arm to determine whether the surgical robot arm reaches a target region, so as to decide whether to re-define a new environmental space and plan another movement path of the surgical robot arm in the new environmental space.

7. The surgical robot arm control system according to claim 6, wherein an end mechanism of the surgical robot arm is equipped with a reference tracking ball, and the processor locates the position of the surgical robot arm based on the reference tracking ball, wherein the processor controls the surgical robot arm according to the movement path of the surgical robot arm, so as to make the end mechanism of the surgical robot arm approach the target region.

8. The surgical robot arm control system according to claim 6, wherein before the surgical robot arm is moved, the processor re-defines a target coordinate point to extend the target coordinate point to a line segment and convert the line segment into a reference target region,
wherein the processor determines whether the reference target region matches the target region to decide whether to control the surgical robot arm according to the movement path of the surgical robot arm.

9. A surgical robot arm control method, comprising:
acquiring spatial coordinate data by a spatial positioning information acquisition unit;
acquiring a panoramic depth image by a depth image acquisition unit;
performing image recognition on the panoramic depth image by a processor to recognize a surgical robot arm;
locating a position of the surgical robot arm by the processor according to the spatial coordinate data;
by the processor, defining an environmental space according to the position of the surgical robot arm and planning a movement path of the surgical robot arm in the environmental space; and
controlling the surgical robot arm by the processor according to the movement path of the surgical robot arm.

10. The surgical robot arm control method according to claim 9, wherein the panoramic depth image comprises at least one tracking ball and an image of the surgical robot arm, and the at least one tracking ball is disposed on a surgical subject,
wherein the step of performing the image recognition on the panoramic depth image comprises:
performing the image recognition on the panoramic depth image by the processor to recognize the at least one tracking ball, the surgical subject, and the surgical robot arm,
wherein the spatial coordinate data comprise a plurality of coordinates of the at least one tracking ball, the surgical subject, and the surgical robot arm.

11. The surgical robot arm control method according to claim 9, wherein the environmental space is centered around an end mechanism of the surgical robot arm, and the environmental space is updated together with a movement of the end mechanism of the surgical robot arm.

12. The surgical robot arm control method according to claim 9, wherein the step of planning the movement path of the surgical robot arm in the environmental space comprises:
training a real path model corresponding to the environmental space by the processor based on a virtual path model through transfer learning to acquire the movement path of the surgical robot arm through the real path model.

13. The surgical robot arm control method according to claim 12, wherein the virtual path model and the real path model are respectively a densely connected convolutional network model.

14. The surgical robot arm control method according to claim 9, further comprising:
after moving the surgical robot arm, recognizing a surgical environment around the surgical robot arm by the processor to determine whether the surgical robot arm reaches a target region, so as to decide whether to re-define a new environmental space and plan another movement path of the surgical robot arm in the new environmental space.

15. The surgical robot arm control method according to claim 14, wherein an end mechanism of the surgical robot arm is equipped with a reference tracking ball,
wherein the step of controlling the surgical robot arm comprises:
locating the position of the surgical robot arm by the processor according to the reference tracking ball; and
controlling the surgical robot arm by the processor according to the movement path of the surgical robot arm to make the end mechanism of the surgical robot arm approach the target region.

16. The surgical robot arm control method according to claim 14, wherein the step of controlling the surgical robot arm comprises:
before moving the surgical robot arm, re-defining a target coordinate point by the processor to extend the target coordinate point to a line segment and convert the line segment into a reference target region; and
determining whether the reference target region matches the target region by the processor, so as to decide whether to control the surgical robot arm according to the movement path of the surgical robot arm.
